(19) United States
(12) Patent Application Publication
(10) Pub. No.: US 2025/0090388 A1
(43) Pub. Date: Mar. 20, 2025

(54) Diaper to Help Potty Train Boys

(71) Applicant: Carol Kleckley, Gainesville, FL (US)

(72) Inventor: Carol Kleckley, Gainesville, FL (US)

(21) Appl. No.: 18/426,857

(22) Filed: Jan. 30, 2024

Related U.S. Application Data
(60) Provisional application No. 63/442,208, filed on Jan. 31, 2023.

(51) Int. Cl. A61F 13/505; A61F 13/56

(52) U.S. Cl. CPC A61F 13/505; A61F 13/565

(57) ABSTRACT

A method includes positioning a first end of a cloth material at an initial position, with the first end being bendable. The method also includes configuring a first connector on a top end of the first end of the cloth material. The first connector is adjacent to an area positioned to receive one or more liquids in set time intervals or random time intervals. The method also includes positioning a second connector adjacent to the area receiving the one or more liquids. The second connector is configured to connect with the first connector to secure the one or more liquids within the area.

### CROSS-REFERENCE TO RELATED APPLICATION

This application claims priority to U.S. Provisional Application No. 63/442,208 filed Jan. 30, 2023, entitled, “Diaper to Help Potty Train Boys,” which is incorporated by reference in its entirety.

### TECHNICAL FIELD

The present inventive concept relates to a diaper to prevent unwanted leak when potty training one or more infants.

### BACKGROUND

Parents of infant toddlers often have to worry about any accidents that occur from their children dispensing liquid on floors or tables and other surfaces. Often, many diapers that hold such infants are not able to prevent such accidents from occurring. As a result, parents of these infants often have to spend excessive time cleaning up these spills.

The user may have difficulty attempting to identify a diaper that can hold the liquids of the infants successfully. Another problem is that the parent/user will have to change the diaper of the infant more intervals than the user would like due to the diaper being unable to hold or absorb the infant's dispensed liquids.

Another issue that users may have to spend an excessive amount of money on diapers and in looking for the right diaper system to absorb and prevent leaks. The time of the user can also be wasted due to the search of looking for the right diaper to place on the infant toddler.

Accordingly, a need exists for a diaper or system that can hold and absorb the liquids dispensed by the toddler throughout the day. This can occur while the toddler is sleeping or awake. Moreover, a need exists for a diaper that can secure the liquid within the diaper at all times to allow the parent to determine when to change the diaper without any accidents occurring.

### SUMMARY

In accordance with the present invention, the present invention relates to a diaper system to potty train infants and prevent leakage of liquid within the diaper system.

In an embodiment of the invention, a method includes positioning a first end of a cloth material at an initial position, with the first end being a bendable material. The method also includes configuring a first connector on a top end of the first end of the cloth material. The first connector is adjacent to an area positioned to receive one or more liquids in set time intervals or random time intervals. The method also includes positioning a second connector adjacent to the area receiving the one or more liquids. The second connector is configured to connect with the first connector to secure the one or more liquids within the area.

The first and second connectors are opened at various time intervals.

An interior region is positioned to hold the one or more liquids in place within the

In an embodiment, a method comprises configuring a first connector at an initial position on a cloth like material. The method also includes placing a receiving pouch to be adjacent to the first connector. The receiving pouch is configured to receive liquids in various time intervals and store the liquids in place in the various time intervals while being in contact with the first connector. The method also includes configuring as second connector at a position opposite to the initial position of the first connector. The second connector is positioned to secure the liquids in place within the receiving pouch.

The method also includes connecting the first connector with the second connector to secure the liquids within the receiving pouch.

The method also includes folding a portion of the cloth to remove the liquids from the receiving pouch.

A system includes a first end of a cloth material positioned at an initial position, with the first end being a bendable material. The system also includes a first connector configured on a top end of the first end of the cloth material. The first connector is adjacent to an area positioned to receive one or more liquids in set time intervals or random time intervals. The system also includes a second connector positioned adjacent to the area receiving the one or more liquids. The second connector is configured to connect with the first connector to secure the one or more liquids within the area.

The first connector is placed in contact with the second connector to hold the one or more liquids within the area.

A second end of the cloth material is folded toward the first end of the cloth material to connect the first connector and the second connector.

### BRIEF DESCRIPTION OF THE FIGURES

FIG. 1 illustrates a portion of a diaper system in accordance with an embodiment of the invention;

FIG. 2 illustrates another view of the diaper system in an embodiment of the invention; and

FIG. 3 illustrates another view of the diaper system in accordance with an embodiment of the invention.

### DETAILED DESCRIPTION

Various embodiments will be described in detail in the specification and in the drawings. Reference numerals represent like parts and assemblies through several views. Reference to various embodiments do not limit the scope of the invention. Further, any additional examples set forth in this specification are not intended to be limiting and merely set forth some of the many possible embodiments for the claimed invention.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

It will be further understood that the terms “comprises,” “comprising,” “includes,” and “including” specify the presence of stated features, steps or components, but do not preclude the presence or addition of one or more features, steps or components. It should also be noted that in some alternative implementations, the functions and acts may occur out of the order noted in the figures. Two figures shown in succession may be executed substantially concurrently or may sometimes be executed in the reverse order.

The term “invention” or “present invention” refers to the invention being applied may be used interchangeably with a diaper system to potty train infants such as infant boys.

### INTRODUCTION

The present invention relates to a diaper or diaper system that will be used to potty train infant toddlers such as infant boys. The diaper system can originally be folded horizontally on both ends. The user such as a parent or guardian can then fold the diaper vertically on both of its ends and place the infant within the diaper system. The parent or guardian places the infant within the diaper with the goal of preventing accidents from the infant accidentally urinating or dispensing liquids onto floors and other surfaces within the home.

The diaper or diaper system will have a fastening system at the front portion of the diaper system. The fastening system will also be in front of a receiving pouch that will receive any liquid that is dispensed by the toddler infant. The receiving pouch will hold all of the liquid that the infant toddler dispenses. The fastening system will be positioned in front of the dispensed liquid and receiving pouch to prevent any liquid from escaping. The fastening system will include a first connector on first end in the front of the diaper system. The fastening system will also include a second connector on a second end in the front of the diaper system.

The user will move the fastening system from the open position to the closed position to ensure that the internal portion of the diaper system holds or absorbs all of the dispensed liquids from the infant toddler. The fastening system will be closed by the user connecting the first connector with the second connector in front of the receiving pouch of the diaper system. The user can keep the fastening system closed for any necessary time interval until the user decides to open the fastening system to empty any liquids within the receiving pouch, or to throw the diaper system away due to overall wetness. The infant toddler can also have the fastening system opened up to urinate in front of the toilet. After finishing the urinating interval, the fastening system can be closed again with no further leaks or spills occurring.

The advantages include no leaks escaping from the diaper system. The fastening system remaining closed allows the parent and guardian to just feel the wetness of the diaper system throughout the day to determine whether to empty the liquids, or to change the diaper system altogether.

The parent or guardian also has the advantage of peace of mind and less expenses spent in cleaning up unexpected spills due to leaks from the diaper. Once the fastening system at the front of the diaper system is closed, the user does not have to worry or anticipate any unexpected leaks/spills from the diaper system.

### Analysis

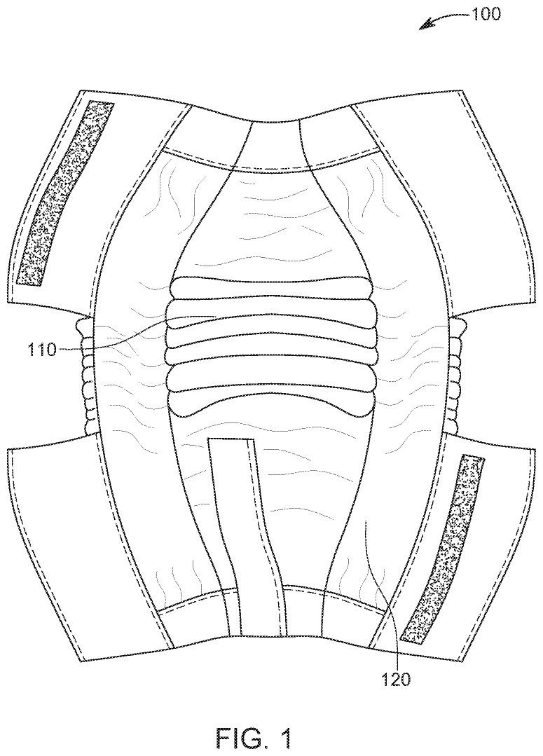

Referring to FIG. 1, a diaper system 100 is illustrated. The diaper system 100 can be placed on a floor or table or any appropriate surface before placing the diaper system 100 on an infant. The diaper system 100 will include a first end 110 and a second end 120. The user can fold diaper system 100 horizontally on the chosen surface before place the infant within the diaper system. In many embodiments, a toddler infant will be involved. first portion of the travelling apparatus 100 is illustrated.

Still referring to FIG. 1, the user will place the infant toddler within the diaper system 100. The user will want to ensure that any accidents from liquids being dispensed by the toddler infant will not spill on the floor. As such, the user will place the infant toddler within the diaper system 100 to prevent the liquids from being dispensed on the floor and other surfaces. The user will then fold vertically both the first end 110 and the second end 120 to wrap around the infant toddler. The diaper system 100 will also have a receiving pouch that will be designed to receive any liquids that are dispensed by the toddler. The receiving pouch will hold all of the liquids that the toddler may dispense at various times throughout the day. Once the diaper system 100 is folded vertically with the first end 110 and second end 120, the user can ensure that no dispensed liquid from the infant toddler will leak out of the diaper system 100. The infant toddler can also have the fastening system opened and urinate in intervals in front of a toilet. The fastening system can also be closed after the infant toddler's urinating intervals, with no spills or leaks occurring.

With respect to FIG. 1, after the first end 110 and second end 120 have been placed around the infant toddler, the user can use the fastening system to position the front portion if the diaper system 100 in place. The front portion of the diaper system 100 will include a button connector on one side of the diaper system 100. Further, the front portion of the diaper system 100 will also include another button connector on another side of the diaper system 100. Both button connectors will be fastened together to secure the front portion of the diaper system 100. The front portion of the diaper system 100 will also include an area or receiving pouch to hold any dispended liquids from the toddler infant. The fastening system being secure on the front portion of the diaper system 100 will ensure that none of the dispended liquids will come out or leak out of the diaper system 100. The user can also use to open the fastening system at various intervals to empty any liquid from the receiving pouch. Nevertheless, the user can also have the fastening system remain closed until deciding to change the diaper on the toddler infant. The user will not have to worry about unexpected spills/leaks when the fastening system at the front of the diaper is closed.

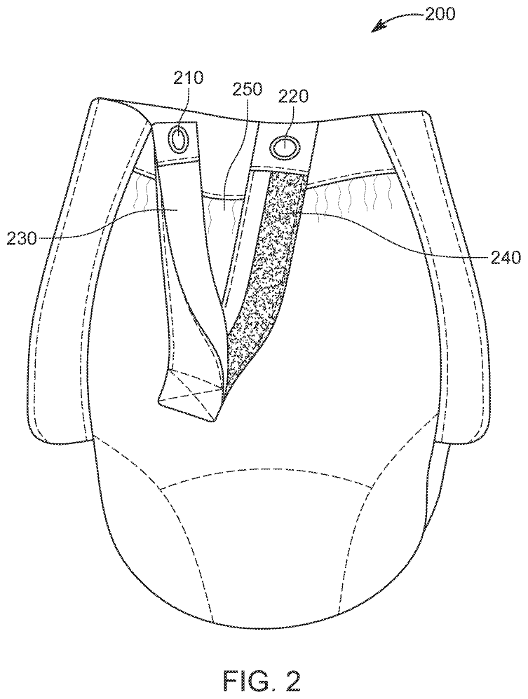

In FIG. 2, another aspect of the diaper system 200 is illustrated. The diaper system 200 can have a fastening system that will hold liquids dispensed by an infant toddler. travelling apparatus 200 is shown. The front portion of the diaper system will include a fastening system that includes a first connector 210 and a second connector 220. The first connector 210 will be positioned on a first end 230 of the diaper system. Further, the second connector 220 will be positioned on a second end 240 of the diaper system 200. The fastening system with the first connector 210 and second connector 220 is positioned in front of a receiving pouch 250. The receiving pouch 250 will be an area that will absorb and hold all liquids dispensed by the toddler infant.

Referring again to FIG. 2, the user will have the infant toddler positioned within the diaper system 200. The user will anticipate that the infant toddler will dispense liquids while within the diaper system 200. The user in set time interval, or multiple time intervals, will close the fastening system on the diaper system 200. The user will connect the first connector 210, which is a button like device, to the second connector 220, wherein the second connector 220 is also a button like device. The user will effectively lock the fastening system in front of the receiving pouch 250. With the first connector 210 being buttoned onto and connected with the second connector 220, the fastening system will remain locked in front of the receiving pouch 250. With the fastening system locked, any dispensed liquid will stay secured within the receiving pouch 250. The interior regions of the receiving pouch 250 will assist the receiving pouch in ensuring that any dispensed liquid from the infant toddler will not escape out of the fastening system. The liquid will be held or absorbed accordingly. The infant toddler can also have the fastening system in front of the receiving pouch 250 opened for urinating intervals in front of a toilet. After the urinating intervals, the fastening system can then be closed, with no spills or leaks occurring.

Still referring to FIG. 2, the user will prevent many accidents from occurring due to dispensed liquid from the infant toddler spill in various places. The fastening system with the first and second connector 210, 220 being connected will ensure that all of the dispensed liquids such as urine and other liquid will remain within the receiving pouch 250. The user can feel when the infant toddler is wet, and determine whether the diaper system 200 of the infant toddler needs to be changed at a later interval.

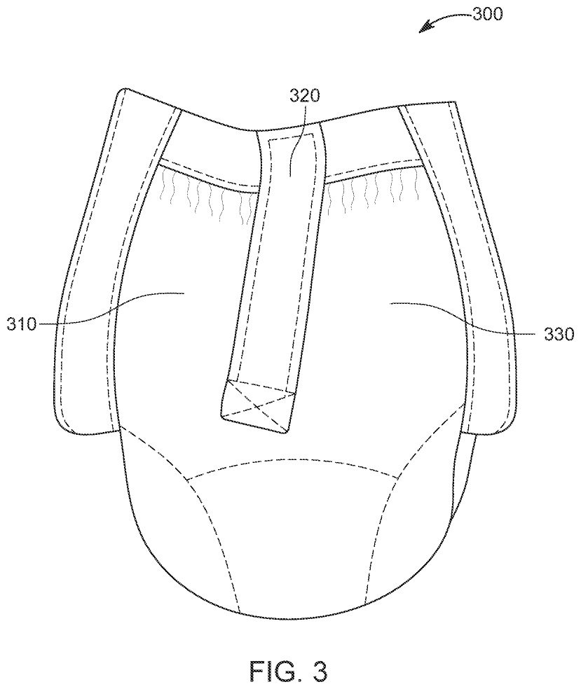

Referring to FIG. 3, another illustration of the diaper system 300 is illustrated. As mentioned above, the user will determine when to close the fastening system of a diaper system 300 once the infant toddler is secured within the diaper system 300. The diaper system 300 will include a first end 310, the fastening system 320, and the second end 330. The first end 310 and the second end 330 will be folded straight and vertically. The fastening system 320 will include the first connector being connected to the second connector. When the fastening system 320 is closed, the first connector is interlocked or connected with the second connector.

Still referring to FIG. 3, the user will determine when the fastening system 320 will be open or closed. The user will secure the infant toddler within the diaper system 300. The user will want to ensure that any liquids which the infant toddler will dispense will not spill onto a table or floor or other surface. The user will thereby close the fastening system 320 to prevent the accidents from occurring. The interior regions within the receiving pouch will ensure that the dispensed liquids stay within the receiving pouch. The liquids will be held or absorbed. In addition, the fastening system 320 remaining closed will also ensure that the dispensed toddler liquids that are not absorbed remain within the receiving pouch. Through various time intervals, the user can periodically check the wetness of the diaper system 300. The user can feel the diaper system 300 at the front and other parts diaper system 300 and determine whether to open the fastening system 320 to dispense any liquids within the receiving pouch. The user will also make the determination whether, based on the wetness of the diaper system 300, whether to change the diaper system 300 of the infant toddler. The infant toddler will also have the fastening system 320 opened for urinating intervals in front of a toilet. After each urinating interval, the fastening system 320 will be closed with no leaks or spills occurring.

With respect to FIG. 3, the advantages of the fastening system 320 remaining closed are accident prevention. No unwanted liquids will be spilled on a table, floor, or other surface. The user is in control to determine how long to keep the diaper system 300 on the infant toddler. At time intervals throughout the day, the user will determine how wet the diaper system 300 is. The user can determine whether to keep the diaper system 300 or remove the diaper system 300 from the toddler. As such, the fastening system 320 remaining closed allows the parent/guardian of the infant toddler to ensure that no spillage of liquids occurs, and that the diaper system 300 is removed from the infant toddler at the appropriate time.

### Summary/Advantages

The user, such as, but not limited to, a parent of an infant toddler can determine the best way to prevent accidents that occur from the infant dispensing liquid on the floor or table or other surface. The user can determine when to put the infant toddler in the diaper system. The user can fold both ends of the diaper system vertically to wrap around the infant toddler. The diaper system will include a first end and second end on the front portion of the diaper system. The diaper system will also include a receiving pouch that is configured to receive all of the dispensed liquids (urine, etc.) from the infant toddler. The center of the front portion of the diaper system will include a fastening system. The fastening system will include a first connector on the first end and a second connector on the second end.

The user will determine when to close the fastening system. When the fastening system is closed, all dispensed liquids from the infant toddler will be prevented from spilling on any surface that is on or near the user. The connection of the first connector to the second connector will prevent the liquids from leaving the fastening system. In addition, the interior regions of the receiving pouch will hold and absorb any of the liquids and also ensure that the liquids do not leave the diaper system and spill onto the floor. In addition, the fastening system can be opened for urinating intervals for the infant toddler in front of a toilet. After each urinating interval, the fastening system can be closed, and any further leak or spill will be prevented.

When the fastening system is closed, the user will ensure that the infant toddler will not spill any liquid due to any unforeseen accidents. The user is also able to check the wetness of the diaper system throughout the day. The user will be able to determine whether to open the fastening system on the diaper system to empty any liquids. In addition, the user is also able to determine whether the diaper system has exceeded a wetness threshold, and change to another diaper. Overall, the user is able to prevent any unwanted accidents, and hold any dispensed liquids within the diaper system. Further, the user is able to change the diaper system on the infant toddler at the right time.

1. A method comprising:
positioning a first end of a cloth material at an initial position, wherein the first end is a bendable material;
configuring a first connector on a top end of the first end of the cloth material, wherein the first connector is adjacent to an area positioned to receive one or more liquids in set time intervals or random time intervals; and
positioning a second connector adjacent to the area receiving the one or more liquids, wherein the second connector is configured to connect with the first connector to secure the one or more liquids within the area.

2. The method of claim 1, further comprising:
opening the first and second connectors at various time intervals.

3. The method of claim 1, further comprising:
positioning an interior region to hold the one or more liquids in place within the area.

4. The method of claim 1, further comprising:
folding a second end of the cloth material to connect the second connector to the first connector.

5. The method of claim 1, further comprising:
configuring the first end of the cloth material to be in contact with a second end of the cloth material to secure the one or more liquids in the area.

6. The method of claim 1, further comprising:
folding the first end of the cloth material over the first and second connectors to secure the one or more liquids in the area.

7. The method of claim 1, further comprising:
removing the one or more liquids from the area at a set time interval.

8. A method comprising:
configuring a first connector at an initial position on a cloth like material;
placing a receiving pouch to be adjacent to the first connector, wherein the receiving pouch is configured to receive liquids in various time intervals, and store the liquids in place in the various time intervals while being in contact with the first connector;
configuring a second connector at a position opposite to the initial position of the first connector, wherein the second connector is positioned to secure the liquids in place within the receiving pouch.

9. The method of claim 8, further comprising:
connecting the first connector with the second connector to secure the liquids within the receiving pouch.

10. The method of claim 8, further comprising:
folding a portion of the cloth to remove the liquids from the receiving pouch.

11. The method of claim 8, further comprising:
position a first and second portion of the cloth to be in contact to secure the liquids within the receiving pouch.

12. The method of claim 8, further comprising:
folding a first and second portion of the cloth to secure the liquids within the receiving pouch.

13. The method of claim 8, further comprising:
positioning a first portion of the cloth over the second portion of the cloth to secure the liquids in the receiving pouch.

14. The method of claim 8, further comprising:
folding a bottom portion of the cloth to place the first connector to be adjacent to the second connector.

15. A system comprising:
a first end of a cloth material positioned at an initial position, wherein the first end is a bendable material;
a first connector configured on a top end of the first end of the cloth material, wherein the first connector is adjacent to an area positioned to receive one or more liquids in set time intervals or random time intervals; and
a second connector positioned adjacent to the area receiving the one or more liquids, wherein the second connector is configured to connect with the first connector to secure the one or more liquids within the area.

16. The system of claim 15, wherein the first connector is placed in contact with the second connector to hold the one or more liquids within the area.

17. The system of claim 15, wherein a second end of the cloth material is folded toward the first end of the cloth material to connect the first connector and the second connector.

18. The system of claim 15, wherein a bottom portion of the cloth material is folded in an upward direction to connect the first connector with the second connector.

19. The system of claim 15, wherein one or more interior regions of the cloth provide support for the first and second connectors as additional liquid is added to the area between the first and second connectors.

20. The system of claim 15, wherein one or more interior regions between the first and second connector secure the one or more liquids for set or random time intervals.
